(19) United States
(12) Patent Application Publication
(10) Pub. No.: US 2006/0264903 A1
(43) Pub. Date: Nov. 23, 2006

(54) SHG catheter and method of use

(76) Inventor: Gregory L. Walker, Whitestown, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/122,650

(22) Filed: May 5, 2005

(51) Int. Cl. A61M 31/00

(52) U.S. Cl. 604/515; 604/275; 604/523

(57) ABSTRACT

A catheter device comprising a cannula and a flexible tip connected to a distal end of the cannula. The cannula has a greater durometer hardness than the flexible tip. In one embodiment, the flexible tip has a durometer hardness of from about 60 Shore A to about 85 Shore A and the cannula has a durometer hardness of from about 65 Shore D to about 80 Shore D.

### FIELD OF THE INVENTION

The present invention relates generally to catheter devices and more particularly to a catheter and method for injecting fluid into a uterus.

### BACKGROUND

Physicians have developed various procedures to exam a woman's uterus and/or uterine lining (i.e., endometrium) for both diagnostic and therapeutic purposes. One such procedure is a sonohysterogram (“SHG”), which involves the examination of a uterine cavity for signs of infertility and/or medical abnormalities, such as unusual bleeding, polyps, fibroids, and internal scarring. Specifically, SHG examinations involve a transvaginal pelvic exam, which is performed in conjunction with an ultrasound of the patient's pelvic region. During the examination, the physician places a speculum into the patient's vagina and inserts a small catheter device into the uterine cavity so that a saline (sterile salt water) solution can be injected. The injection of the saline solution into the uterine cavity causes the endometrial walls of the uterus to separate and enhances the visibility of any abnormal masses which may be present within the uterus. Moreover, the saline solution also assists in contrasting the internal features of the uterus so that any medical abnormalities can be detected.

Many of the devices used for the above described procedure require intricate and complicated catheter arrangements that have multiple pieces and and/or components. These complicated devices are often difficult to use and expensive to manufacture. Moreover, many of the devices have ineffective sealing components, thereby allowing the saline solution to leak back out of the uterus once injected. Because of these problems, many patients are subjected to unnecessary pain and/or some level of discomfort while undergoing these procedures.

As such, it is desirable to have a catheter device that overcomes all of the disadvantages noted above.

### SUMMARY OF THE INVENTION

The present invention provides a catheter and method for injecting fluid into a uterus.

In one exemplary embodiment thereof, the present invention provides a catheter comprising a cannula and a flexible tip connected to a distal end of the cannula. According to this embodiment, the cannula has a greater durometer hardness than the flexible tip.

In another exemplary embodiment thereof, the present invention provides a method of injecting fluid into a uterine cavity of a patient. The method comprises providing a catheter having a cannula and a flexible tip connected to a distal end of the cannula, wherein the cannula has a greater durometer hardness than the flexible tip. The face of the patient's cervix is probed with the flexible tip and advanced through the cervical opening and into the uterine cavity. A seal is formed over the cervical opening with a flange member located at a proximal end of the flexible tip and fluid is ejected from the flexible tip and into the uterine cavity. During the procedure, the cannula is adapted to resist flexing while the flexible tip is capable of flexing.

In specific exemplary embodiments, the durometer hardness of the cannula is from about 65 Shore D to about 80 Shore D, and the durometer hardness of the flexible tip is from about 60 Shore A to about 85 Shore A.

### BRIEF DESCRIPTION OF DRAWINGS

The above-mentioned aspects of the present invention and the manner of obtaining them will become more apparent and the invention itself will be better understood by reference to the following description of the embodiments of the invention taken in conjunction with the accompanying drawings, wherein:

FIG. 1 is a perspective view of an exemplary embodiment of a catheter device of the present invention held in the hands of a physician;

FIG. 2a is a perspective view of an exemplary embodiment of a connector component for the catheter device of the present invention;

FIG. 2b is a perspective view of an exemplary embodiment of a cannula for the catheter device of the present invention;

FIG. 2c is a perspective view of an exemplary embodiment of a flexible tip for the catheter device of the present invention;

FIG. 3 is a schematic view of the catheter device of the present invention inserted into a uterine cavity;

FIG. 4 is a perspective view of the flexible tip for the catheter device of the present invention shown with the tip pushed forward against a surface;

FIG. 5 is a perspective view of the flexible tip for the catheter device of the present invention shown with the tip pushed forward through a surface opening;

FIG. 6 is a perspective view of the flexible tip for the catheter device of the present invention sealing a surface opening after being pushed through the surface opening; and

FIG. 7 is a perspective view of the flexible tip for the catheter device of the present invention shown with the tip bending in more than one direction.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

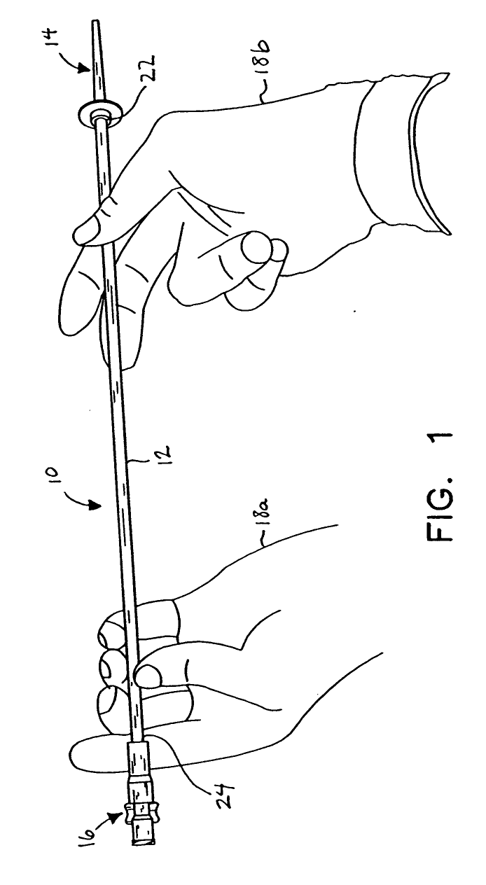

Referring now to FIG. 1, the catheter device 10 of one exemplary embodiment includes a cannula 12, a flexible tip 14 and a connector component 16 shown held in a physician's hands 18a-b. According to this embodiment, the flexible tip 14 is connected to the distal end 22 of the cannula 12 and the connector component 16 is attached to the cannula's proximal end 24. It should be understood that the flexible tip 14 and connector component's 16 connections with the cannula 12 may be permanent or allow disconnection. In exemplary embodiments, cannula 12 may be attached to any of a plurality of differently sized or shaped flexible tips 14 and/or connector components 16, as is further explained below. Alternatively, the cannula 12 may be securely connected to the flexible tip 14 and/or the connector component 16, such that the catheter device 10 functions as a unitary device.

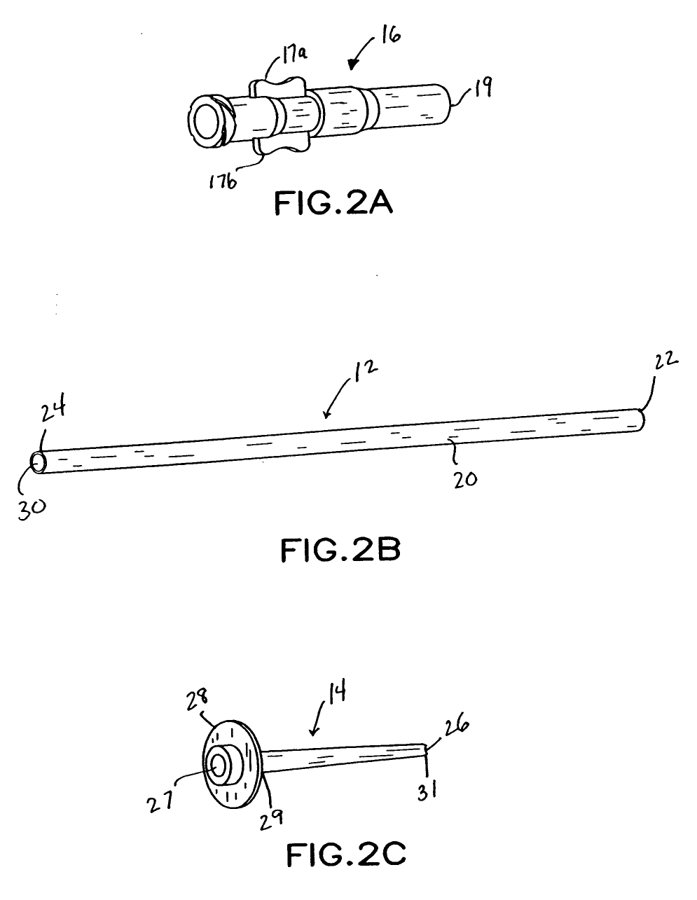

Turning now to FIG. 2a, a perspective view of the connector component 16 is depicted according to the present invention. The connector component 16 has a pair of winged ears 17a, 17b adapted to lock to a fluid delivery device (not shown). Moreover, the connector component 16 is also attached to the cannula 12 at the cannula's proximal end 24. More particularly, the cannula's proximal end 24 is inserted into connector opening 19 and securely attached by any fastening means known in the art. Exemplary connector components 16 according to the present invention include, but are not limited to, luer connector devices. Luer connector devices are well known in the field of medicine and are typically used for coupling a liquid or gas source to a catheter line or other medical device. As is appreciated by those skilled within the relevant art, luer connector devices useful according to the present invention may be female or male in orientation and may function as luer-locking devices, luer-slip connection devices or the like.

In an exemplary embodiment according to the present invention, a fluid delivery device is connected to the connector component 16 for delivery of fluid into a patient's uterine cavity. According to this embodiment, fluid is delivered into the uterus by way of the catheter device's 10 cannula 12. As depicted in FIG. 2b, the cannula 12 generally includes a proximal end 24, a distal end 22, and an outer body 20 extending between the proximal and distal ends 24, 22 that defines a central lumen 30 disposed therein. The lumen 30 is adapted to allow a fluid, such as a saline solution, to pass through the cannula 12 to be delivered into a patient's uterus. Delivery of fluid into the lumen 30 may be accomplished by any known delivery device, including but not limited to a syringe, catheter, hubbed needle, IV tube, cylinder fluid delivery device or the like. According to this embodiment, fluid passes through the lumen 30 of the cannula 12 and enters the flexible tip 14 before ultimately being ejected out of the flexible tip 14 and into the patient's uterus.

In certain exemplary embodiments according to the present invention, the central lumen 30 of the cannula comprises a single lumen structure. More particularly, unlike other catheter arrangements requiring more than one cannula and/or lumen structure, the catheter device 10 provides a single lumen arrangement. Because of the effective and simplistic arrangement of these devices, physicians can easily and quickly perform uterine exams with minimal discomfort to the patient. In further exemplary embodiments according to the present invention, the central lumen 30 of the cannula extends throughout the flexible tip 14. According to this exemplary embodiment, the catheter device 10 has a single lumen extending through the cannula 12 and flexible tip 14 structures.

The flexible tip 14 of the illustrated embodiment is generally depicted in FIG. 2c. The tip has a flanged member 28 at its proximal end 29 and an opening 26 at its distal end 31. Moreover, the flexible tip 14 is adapted to attach to the cannula 12 at the cannula's distal end 22. More particularly, the cannula's distal end 22 is inserted into flexible tip opening 27 and securely attached by any fastening means known in the art. The opening 26 of the flexible tip 14 is adapted to allow the release of fluid into the uterine cavity. As such, the flexible tip 14 is hollow throughout, forming a single lumen. As the flexible tips of the present invention must be flexible, it is desirable that they be produced from an elastomeric material. Known elastomeric materials capable of being used to manufacture the flexible tip according to the present invention include, but are not limited to, thermoplastic vinyl materials, such as polyvinyl chlorides (PVC). Those skilled in the art will appreciate, however, that other flexible materials in addition to elastomeric materials may alternatively be used to manufacture the flexible tip 14 while still staying within the scope of the present invention.

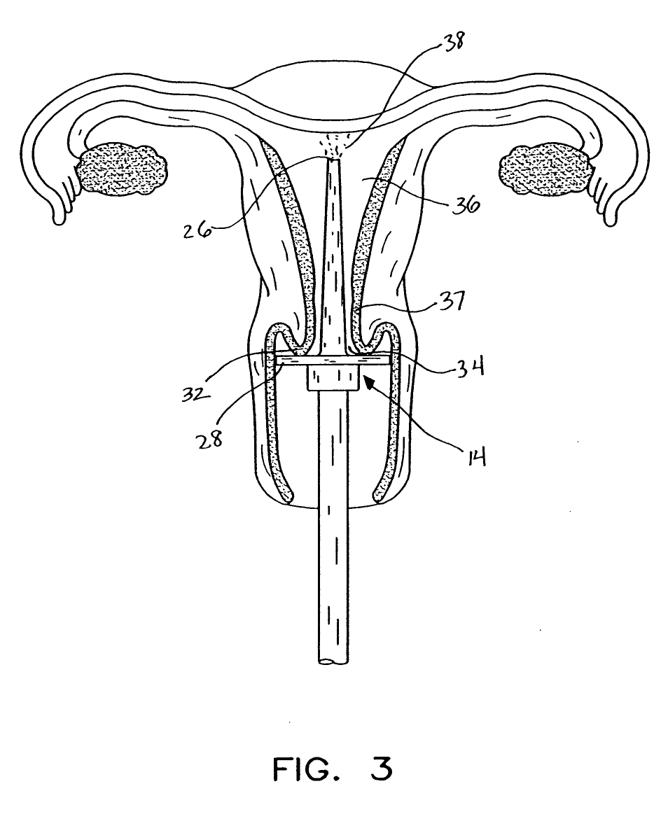

Still referring to FIG. 2c, the flanged member 28 of the flexible tip 14 is adapted to attach to the distal end 22 of the cannula and engage a cervical opening of a uterine cavity during a physical examination. More particularly, in exemplary embodiments according to the present invention, the flexible tip 14 is adapted to engage and pass through the cervical opening of a uterus at the direction of a physician. According to this embodiment, and with reference to FIG. 3, the flexible tip 14 of the catheter device 10 probes the face of a patient's cervix 32 until it locates the cervical opening 34 of the uterine cavity 36, whereupon it gently advances through (dilates) the cervical opening 34 without piercing the cervical tissue 37 and/or creating a false passage. To locate the cervical opening 34, the flexible tip 14 is gently flexed along the face of the patient's cervix by the physician. This is accomplished by the physician using the rigid cannula 12 as a guide to direct the flexible tip 14 as it is pressed along the surface of the patient's cervix 32 to locate the cervical opening 34. Once the flexible tip 14 has entered the uterine cavity 36, the physician is able to eject a fluid (saline) solution 38 from the flexible tip 14 and into the uterine cavity 36. At this time, the flanged member 28 of the flexible tip 14 is positioned to abut the cervical opening 34 in such a manner that it closes off the cervical opening 34 and forms a cervical seal. By creating a seal outside the cervical opening 34 with the flanged member 28, leakage of injected fluid 38 back out of the uterine cavity 36 is minimized and/or eliminated.

It should be understood and appreciated that while inserting the flexible tip into the cervical opening, the physician may utilize a light magnifying instrument such as a colposcope to visualize the cervical opening while advancing the tip into the uterine cavity for injecting the fluid.

The cannula 12 remains relatively rigid or inflexible as it is being positioned and advanced into the patient's uterine cavity (i.e., the cannula resists flexing during the procedure). The flexible tip 14, on the other hand, is adapted to exhibit some flexibility during the procedure so that it can gently probe the patient's cervical opening and advance without pain, tearing and/or discomfort to the individual undergoing the procedure. The rigid and flexible characteristics of the respective cannula and flexible tip according to the catheter devices of the present invention are explained in more detail below.

Regarding the dimensional aspects of the catheter device, the cannula 12 generally has a diameter ranging from about 3 to about 5 millimeters and a length of from about 7 to about 10 inches. Moreover, the flexible tip 14, has a diameter ranging from about 1.5 to about 3.5 millimeters and a length of from about 20 to about 40 millimeters. As illustrated in FIG. 2c, for instance, the flexible tip 14 may taper in diameter from one end to the other, or alternatively, the flexible tip may have one constant diameter. Those skilled in the relevant art will appreciate that other dimensional measurements may be used for the catheter devices depending on the circumstances surrounding the patient to be examined and/or the procedure to be conducted. As such, the dimensional measurements provided herein are not intended to be limiting in nature.

With respect to the hardness of the catheter devices, the flexible tip 14 generally has a durometer hardness of from about 60 Shore A to about 85 Shore A. Moreover, the durometer hardness of the cannula 12 is typically from about 65 Shore D to about 80 Shore D. In a further exemplary embodiment, the flange member 28 of the flexible tip 14 has the same durometer hardness as the entire flexible tip structure. As is generally known within the field of plastics, hardness may be determined by the Shore (Durometer) test, which measures the resistance of a plastic material (elastomer) toward indentation. Shore hardness is typically categorized on either a Shore A or Shore D scale by using a durometer apparatus, which penetrates the sample material. The Shore A scale is used for “softer” rubbers, while the Shore D scale is used for “harder” ones.

In exemplary embodiments according to the present invention, the cannula 12 has a hardness greater than the hardness of the flexible tip 14. The rigid structure of the cannula 12 allows the physician to easily and accurately position the flexible tip 14 of the catheter device 10 outside of the patient's cervix 32 during an examination. Moreover, the hardness of the cannula 12 relative to the flexible tip 14 also offers the physician some “pushability” of the catheter device 10 upon conducting an examination.

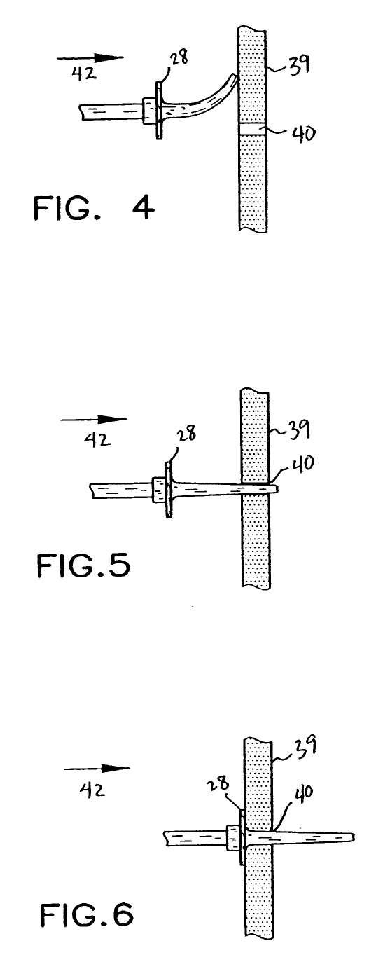

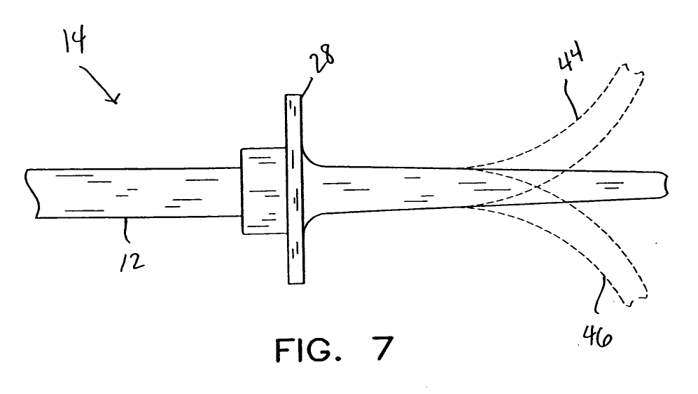

While the flexible tip 14 according to the present invention is adapted to be advanced through a patient's cervix 32 during an examination, the tip 14 material nevertheless requires a significant amount of flexibility. The flexibility of the flexible tip 14 is generally depicted in FIGS. 4-7. As depicted in FIG. 4, when the flexible tip 14 is advanced in a forward motion (as indicated by arrow 42) against a solid surface 39, the flexible tip 14 is adapted to bend in shape. In addition to its flexibility, the tip 14 must also have enough adaptability to allow the physician to gently “push” its structure through a surface opening 40, as indicated in FIG. 5. Once the flexible tip 14 is completely pushed through the surface opening 40, the flanged member 28 of the tip is adapted to provide a seal over the surface opening 40 to prevent a fluid from exiting out of the opening (see FIG. 6). As depicted in FIG. 7, the flexibility of the flexible tip 14 is not limited directionally, and can be bent in any direction desired (as indicated by phantom lines 44 and 46).

While an exemplary embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A catheter comprising:
a cannula; and
a flexible tip connected to a distal end of the cannula, the cannula having a greater durometer hardness than the flexible tip.

2. The catheter of claim 1, wherein the flexible tip comprises an opening to release fluid.

3. The catheter of claim 1, wherein the flexible tip comprises a flange member adapted to seal an opening.

4. The catheter of claim 3, wherein the flange member is located at a proximal end of the flexible tip.

5. The catheter of claim 1, wherein the flexible tip is securely connected to the distal end of the cannula.

6. The catheter of claim 1, wherein the flexible tip comprises thermoplastic vinyl.

7. The catheter of claim 6, wherein the thermoplastic vinyl comprises polyvinyl chloride.

8. The catheter of claim 1, wherein the durometer hardness of the flexible tip is from about 60 Shore A to about 85 Shore A.

9. The catheter of claim 1, wherein the durometer hardness of the cannula is from about 65 Shore D to about 80 Shore D.

10. The catheter of claim 1, wherein the cannula comprises a central lumen.

11. The catheter of claim 10, wherein the central lumen comprises a single lumen.

12. The catheter of claim 1, wherein the flexible tip comprises a central lumen.

13. The catheter of claim 12, wherein the central lumen comprises a single lumen.

14. The catheter of claim 1, wherein the cannula has a diameter of from about 3 to about 5 millimeters.

15. The catheter of claim 1, wherein the cannula has a length of from about 7 to about 10 inches.

16. The catheter of claim 1, wherein the flexible tip has a diameter of from about 1.5 to about 3.5 millimeters.

17. The catheter of claim 1, wherein the flexible tip has a length of from about 20 to about 40 millimeters.

18. The catheter of claim 1, wherein the catheter further comprises a connector component.

19. The catheter of claim 18, wherein the connector component is a luer connector.

20. A catheter comprising:
a cannula; and
a flexible tip connected to a distal end of the cannula, the flexible tip including a flange member adapted to seal an opening;
wherein the cannula has a greater durometer hardness than the flexible tip and the flange member has the same durometer hardness as the flexible tip.

21. The catheter of claim 20, wherein the flexible tip further comprises an opening to release fluid.

22. The catheter of claim 20, wherein the durometer hardness of the flexible tip is from about 60 Shore A to about 85 Shore A.

23. The catheter of claim 20, wherein the durometer hardness of the cannula is from about 65 Shore D to about 80 Shore D.

24. The catheter of claim 20, wherein the flange member is located at a proximal end of the flexible tip.

25. The catheter of claim 20, wherein the flexible tip is securely connected to the distal end of the cannula.

26. A method of injecting fluid into the uterine cavity of a patient, comprising:
(a) providing a catheter having a cannula and a flexible tip connected to a distal end of the cannula, the cannula having a greater durometer hardness than the flexible tip;
(b) probing the face of the patient's cervix with the flexible tip;
(c) advancing the flexible tip through the cervical opening and into the uterine cavity;
(d) sealing the cervical opening with a flange member located at a proximal end of the flexible tip;
(e) ejecting fluid from the flexible tip and into the uterine cavity; and
(f) resisting flexing the cannula during the procedure while flexing the flexible tip.

27. The method of claim 26, wherein the flexing comprises bending the flexible tip in more than one direction.

28. The method of claim 26, wherein step (b) comprises locating the patient's cervical opening by pressing the flexible tip against the cervix so that it flexes along the face of the cervix.

29. The method of claim 26, wherein the durometer hardness of the flexible tip is from about 60 Shore A to about 85 Shore A.

30. The method of claim 26, wherein the durometer hardness of the cannula is from about 65 Shore D to about 80 Shore D.

31. The method of claim 26, wherein the flange member has the same durometer hardness as the flexible tip.

32. The method of claim 26, wherein the cannula comprises a central lumen.

33. The method of claim 32, wherein the central lumen comprises a single lumen.

34. The method of claim 32, wherein the central lumen extends through the flexible tip.
